(19) United States
(12) Patent Application Publication
(10) Pub. No.: US 2009/0130660 A1
(43) Pub. Date: May 21, 2009

(54) Single Nucelotide Polymorphism (SNP)

(76) Inventors: Malek Faham, San Mateo County, CA (US); Soren Germer, Essex County, NJ (US); Hywel Bowden Jones, San Francisco County, CA (US); Delphine Lagarde, Hegenheim (FR); Mitchell Lee Martin, Essex County, NJ (US); Martin Emilio Moorhead, San Mateo County, CA (US); Erik Roy Rasmussen, Suffolk, NY (US); Brian Kent Rhees, San Joaquin County, CA (US); James Andrew Rosinski, Essex County, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/792,772

(22) PCT Filed: Dec. 5, 2005
(86) PCT No.: PCT/EP2005/012987

§ 371 Date: May 7, 2008

Related U.S. Application Data
(60) Provisional application No. 60/635,554, filed on Dec. 13, 2004.
(60) Provisional application No. 60/714,302, filed on Sep. 6, 2005.

(51) Int. Cl. C12Q 1/68

(52) U.S. Cl. 435/6

(57) ABSTRACT

Association of Type 2 diabetes with single nucleotide polymorphisms and haplotypes are disclosed. Also disclosed are diagnostic applications in identifying those who have Type 2 diabetes or are at risk of developing Type 2 diabetes, and discovery of therapeutic agents and methods of treatment.

Diabetes mellitus, a metabolic disease in which carbohydrate utilization is reduced and lipid and protein utilization is enhanced, is caused by an absolute or relative deficiency of insulin. In the more severe cases, diabetes is characterized by chronic hyperglycemia, glycosuria, water and electrolyte loss, ketoacidosis and coma. Long term complications include development of neuropathy, retinopathy, nephropathy, generalized degenerative changes in large and small blood vessels and increased susceptibility to infection. The most common form of diabetes is Type 2, non-insulin-dependent diabetes that is characterized by hyperglycemia due to impaired insulin secretion and insulin resistance in target tissues. Both genetic and environmental factors contribute to the disease. For example, obesity plays a major role in the development of the disease. Type 2 diabetes is often a mild form of diabetes mellitus of gradual onset.

The health implications of Type 2 diabetes are enormous. In 1995, there were 135 million adults with diabetes worldwide. It is estimated that dose to 300 million will have diabetes in the year 2025. (King H., et al., Diabetes Care, 21(9): 1414-1431 (1998)).

Type 2 diabetes has been shown to have a strong familial transmission: 40% of monozygotic twin pairs with Type 2 diabetes also have one or several first degree relatives affected with the disease. Barnett et al. 20 Diabetologia 87-93 (1981). In the Pima Indians, the relative risk of becoming diabetic is increased twofold for a child born to one parent who is diabetic, and sixfold when both parents are affected Knowler, W. C., et al. Genetic Susceptibility to Environmental Factors. A Challenge for Public Intervention 67-74 (Almquist & Wiksele International: Stockholm, 1988). Concordance of monozygotic twins for Type 2 diabetes has been observed to be over 90%, compared with approximately 50% for monozygotic twins affected with Type I diabetes. Barnett, A. H., et al. 20(2) Diabetologia 87-93 (1981). Non-diabetic twins of Type 2 diabetes patients were shown to have decreased insulin secretion and a decreased glucose tolerance after an oral glucose tolerance test Barnett, A H., et al. 282 Brit, Med. J. 1656-1658 (1981).

The high prevalence of the disease and increasing population affected shows an ummet medical need to define other genetic factors involved in Type 2 diabetes and to more precisely define the associated risk factors. Also needed are diagnostic assays to identify the propensity to develop Type 2 diabetes and therapeutic agents for prevention and treatment of the disease.

A nucleic acid sequence at which more than one sequence is possible in a population (either a natural population or a synthetic population, e.g., a library of synthetic molecules) is referred to herein as a “polymorphic site.” Polymorphic sites can allow for differences in sequences based on substitutions, insertions, or deletions. Such substitutions, insertions, or deletions can result in frame shifts, the generation of premature stop codons, the deletion or addition of one or more amino acids encoded by a polynucleotide, alter splice sites, and affect the stability or transport of MRNA. Where a polymorphic site is a single nucleotide in length, the site is referred to as a single nucleotide polymorphism (“SNP”).

SNPs are the most common form of genetic variation responsible for differences in disease susceptibility and drug response. SNPs can directly contribute to or, more commonly, serve as markers for many phenotypic endpoints such as disease risk or the drug response differences between patients.

Identification of these genetic factors can lead to diagnostic methods, reagents and reagent kits for the identification of individuals who have a propensity to develop certain diseases.

The instant invention concerns the identification of genetic factors that predispose individuals to diabetes, with a focus on candidate genes and specifically, nucleic acid fragments of genes having single nucleotide polymorphisms (“SNPs”) which are amenable to diagnostic and therapeutic intervention.

In certain embodiments, the invention provides isolated polynucleotides containing SNPs located within sequences selected from the group consisting of sequences identified by Sequence Identification Numbers (“SEQ. ID. NOS.”) 1-7 and the complements of the sequences identified by SEQ. ID. NOS.: 1-7 as well as vectors, recombinant host cells, transgenic animals, and compositions containing such polynucleotides. The invention also provides methods of diagnosing a susceptibility to Type 2 diabetes in an individual, by detecting one or more at-risk alleles of SNPs associated with Type 2 diabetes. In addition, the invention provides methods of diagnosing a susceptibility to Type 2 diabetes in an individual by detecting one or more haplotypes associated with Type 2 diabetes.

Also contemplated by the invention are methods of identifying agents which can alter the course of the disease as well as the agents themselves and pharmaceutical compositions comprising these agents.

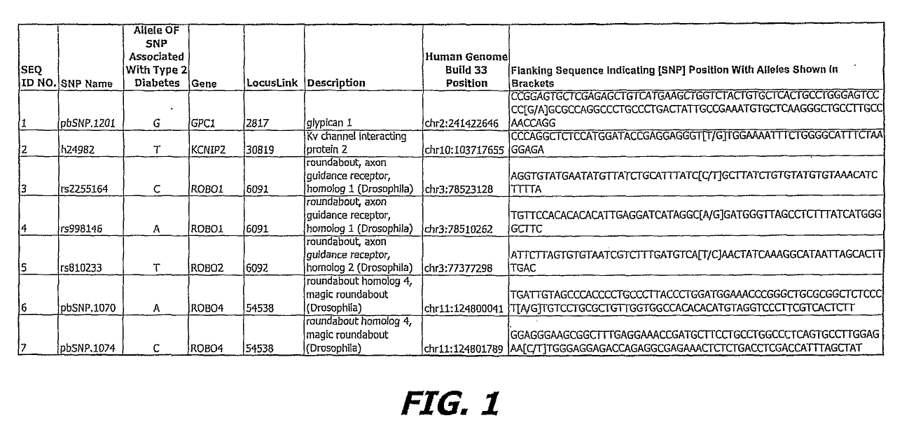

FIG. 1 shows SEQ. ID. NOS.: 1-7 with SNPs indicated by brackets within each sequence. The allele of each SNP that is associated with Type 2 diabetes is shown in a separate column.

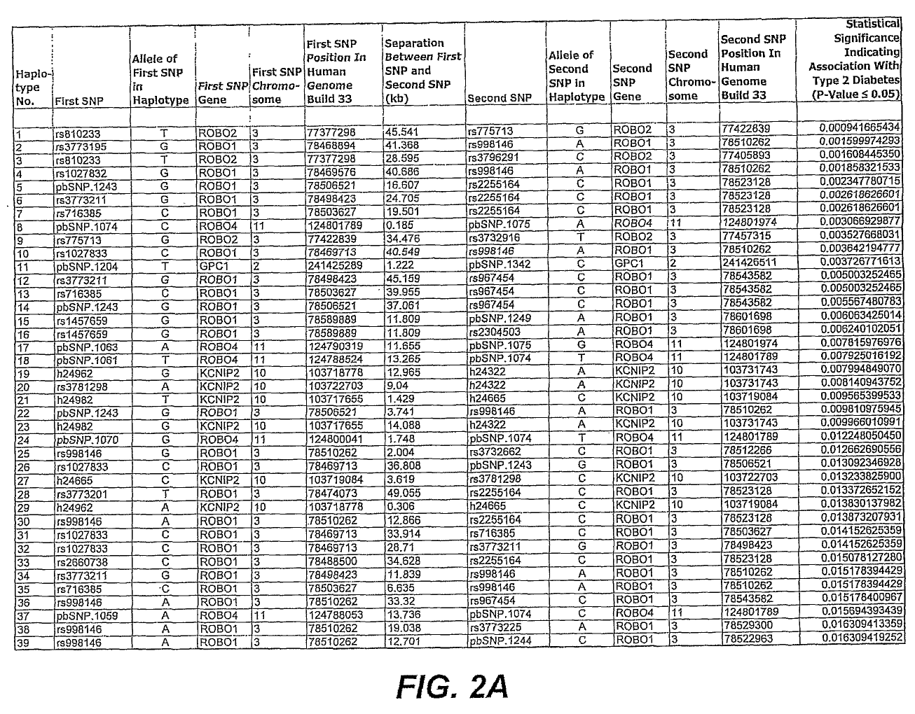

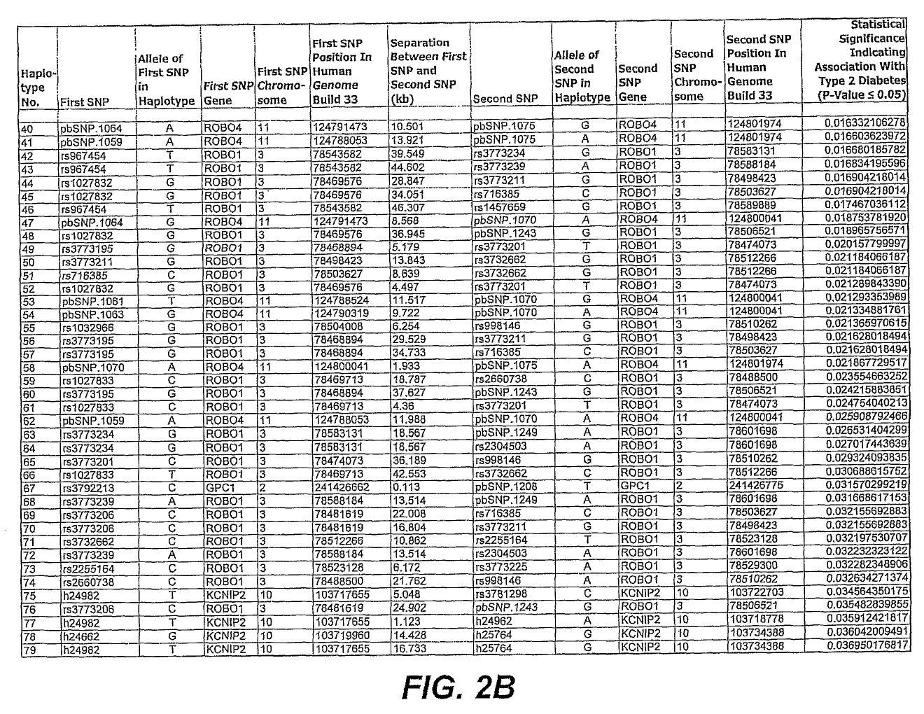

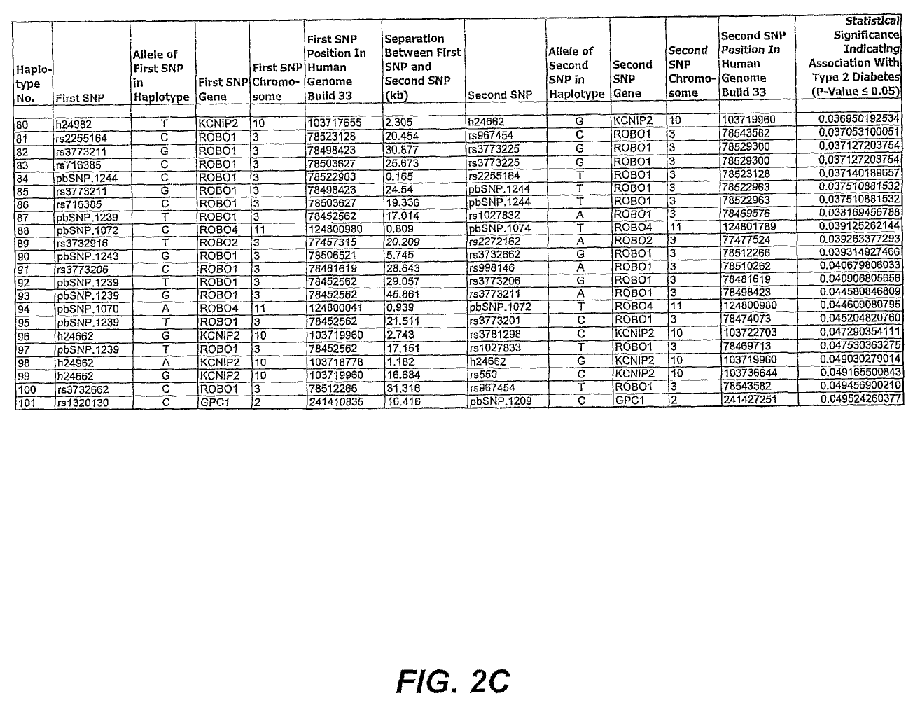

FIGS. 2A-2C (collectively referred to herein as “FIG. 2”) show haplotypes associated with Type 2 diabetes.

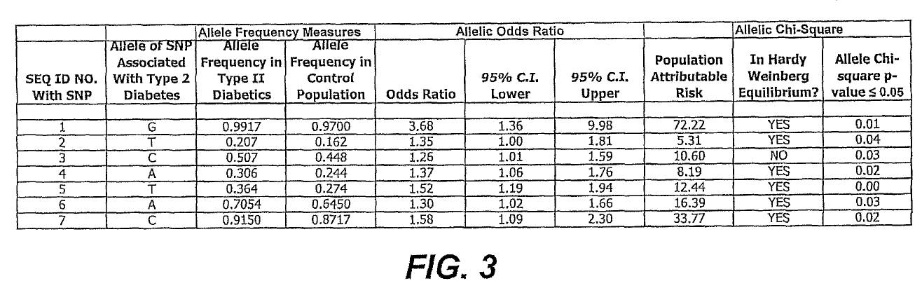

FIG. 3 shows how much each at-risk allele identified for each SNP in FIG. 1 is associated with Type 2 diabetes (significance at p≦0.05) based upon the allelic chi-square association test.

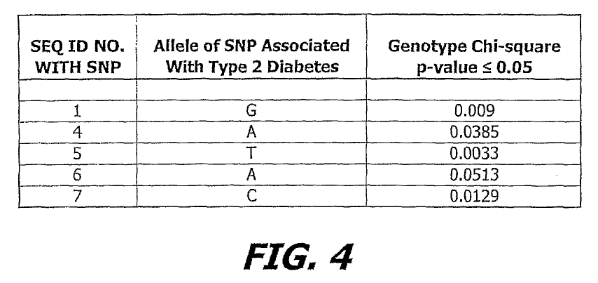

FIG. 4 shows how much each at-risk allele identified for each SNP in FIG. 1 is associated with Type 2 diabetes (significance at p≦0.05) based upon the genotypic chi-square association test.

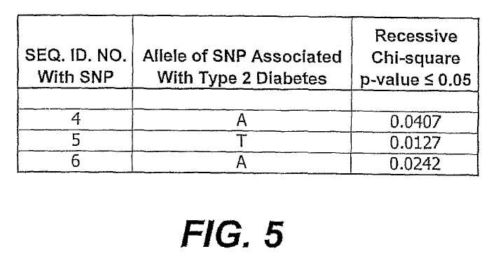

FIG. 5 shows how much each at-risk allele identified for each SNP in FIG. 1 is associated with Type 2 diabetes (significance at p≦0.05) based upon the chi-square test for recessive effects.

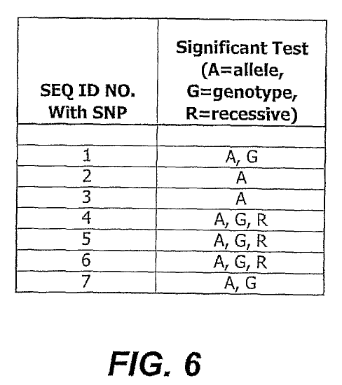

FIG. 6 provides a summary of the SNPs found to be associated with Type 2 diabetes using allelic association, genotypic association and/or the chi-square test for recessive effects.

Single nucleotide polymorphisms, the most frequent DNA sequence variations in the human genome, gain more and more importance for a wide range of biological and biomedical applications. SNPs are used to explore the evolutionary history of human populations and to analyze forensic samples. SNPs also play a major role in genetic analysis. In addition, pharmacogenetics utilizes these DNA variations to elucidate genetic factors that underlie different drug efficacies or adverse events. Finally, SNPs are thought to help identify genes that are involved in complex diseases.

The present invention relates to the identification of specific loci or single nucleotide polymorphisms (SNPs) that are specifically identified to be phenotypically associated with Type 2 diabetes. As a consequence, intervention can be prescribed to such individuals before symptoms of the disease present, e.g., dietary changes, exercise and/or medication. Identification of genes implicated in Type 2 diabetes locus can pave the way for a better understanding of the disease process, which in turn can lead to improved diagnostics and therapeutics.

Genes thought to be implicated in Type 2 diabetes were analyzed to identify SNPs. Nucleic acid sequences containing the SNPs were then genotyped in diabetic cases and matched controls. Statistical analysis was then performed to find association with Type 2 diabetes in analysis of control and diabetic populations. After the analysis of 1,769 SNPs in 186 genes, certain SNPs were found to be statistically associated with Type 2 diabetes (p≦0.05).

The term “SNP” refers to a single nucleotide polymorphism at a particular position in the human genome that varies among a population of individuals. As used herein, a SNP may be identified by its name or by location within a particular sequence. The SNPs identified in the SEQ. ID. NOS. of FIG. 1 are indicated by brackets. For example, the SNP “[G/A]” in SEQ. ID. NO.: 1 of FIG. 1 indicates that the nucleotide base (or the allele) at that position in the sequence may be either guanine or adenine. The allele associated with Type 2 diabetes in FIG. 1 (e.g., a guanine in SEQ. ID. NO.: 1) is indicated in a separate column. The nucleotides flanking the SNP for each SEQ. ID. NO. in FIG. 1 are the flanking sequences which are used to identify the location of the SNP in the genome.

As used herein, the nucleotide sequences disclosed by the SEQ. ID. NOS. of the present invention encompass the complements of said nucleotide sequences. In addition, as used herein, the term “SNP” encompasses any allele among a set of alleles. The term “allele” refers to a specific nucleotide among a selection of nucleotides defining a SNP.

The term “minor allele” refers to an allele of a SNP that occurs less frequently within a population of individuals than the major allele.

The term “major allele” refers to an allele of a SNP that occurs more frequently within a population of individuals than the minor allele.

The term “at-risk allele” refers to an allele that is associated with Type 2 diabetes. FIG. 1 and FIGS. 3-5 show a number of at-risk alleles of the present invention. The term “haplotype” refers to a combination of particular alleles from two or more SNPs.

The term “at-risk haplotype” refers to a haplotype that is associated with Type 2 diabetes. FIG. 2 shows a number of at-risk haplotypes of the present invention.

The term “polynucleotide” refers to polymeric forms of nucleotides of any length. The polynucleotides may contain deoxyribonucleotides, ribonucleotides, and/or their analogs. Polynucleotides may have any three-dimensional structure including single-stranded, double-stranded and triple helical molecular structures, and may perform any function, known or unknown. The following are non-limiting embodiments of polynucleotides: a gene or gene fragment, exons, introns, mRNA, tRNA, rRNA, short interfering nucleic acid molecules (siNA), ribozymes, cDNA, recombinant polynucleotides, branched polynucleotides, plasmids, vectors, isolated DNA of any sequence, isolated RNA of any sequence, nucleic acid probes, and primers. A polynucleotide may also comprise modified nucleic acid molecules, such as methylated nucleic acid molecules and nucleic acid molecule analogs.

A “substantially isolated” or “isolated” polynucleotide is one that is substantially free of the sequences with which it is associated in nature. By substantially free is meant at least 50%, at least 70%, at least 80%, or at least 90% free of the materials with which it is associated in nature. An “isolated polynucleotide” also includes recombinant polynucleotides, which, by virtue of origin or manipulation: (1) are not associated with all or a portion of a polynucleotide with which it is associated in nature, (2) are linked to a polynucleotide other than that to which it is linked in nature, or (3) does not occur in nature.

The term “hybridizes under stringent conditions” is intended to describe conditions for hybridization and washing under which nucleotide sequences at least 60%, 65%, 70%, 75%, 80%, 85%, 90%, 95%, or 98% identical to each other typically remain hybridized to each other. Such stringent conditions are known to those skilled in the art and can be found in Current Protocols in Molecular Biology, John Wiley & Sons, N.Y (1989), 6.3.1-6.3.6. A non-limiting example of stringent hybridization conditions are hybridization in 6× sodium chloride/sodium citrate (SSC) at about 45° C., followed by one or more washes in 0.2×SSC, 0.1% SDS at 50-65° C.

The term “vector” refers to a DNA molecule that can carry inserted DNA and be perpetuated in a host cell. Vectors are also known as cloning vectors, cloning vehicles or vehicles. The term “vector” includes vectors that function primarily for insertion of a nucleic acid molecule into a cell, replication vectors that function primarily for the replication of nucleic acids, and expression vectors that function for transcription and/or translation of the DNA or RNA. Also included are vectors that provide more than one of the above functions.

A “host cell” includes an individual cell or cell culture which can be or has been a recipient for vector(s) or for incorporation of nucleic acid molecules and/or proteins. Host cells include progeny of a single host cell, and the progeny may not necessarily be completely identical (in morphology or in total DNA complement) to the original parent due to natural, accidental, or deliberate mutation. A host cell includes cells transfected with the polynucleotides of the present invention. An “isolated host cell” is one which has been physically dissociated from the organism from which it was derived.

The terms “individual,” “host,” and “subject” are used interchangeably herein to refer to a vertebrate, preferably a mammal, more preferably a human.

The terms “transformation,” “transfection,” and “genetic transformation” are used interchangeably herein to refer to the insertion or introduction of an exogenous polynucleotide into a host cell, irrespective of the method used for the insertion, for example, lipofection, transduction, infection, electroporation, CaPO₄ precipitation, DEAE-dextran, particle bombardment, etc. The exogenous polynucleotide may be maintained as a non-integrated vector, for example, a plasmid, or alternatively, may be integrated into the host cell genome. The genetic transformation may be transient or stable.

The present invention employs, unless otherwise indicated, conventional techniques of molecular biology (including recombinant techniques), microbiology, cell biology, biochemistry and immunology, which are within the skill of the art.

As used herein, the singular form of any term can alternatively encompass the plural form and vice versa.

All publications and references cited herein are incorporated by reference in their entirety for any purpose.

The present invention provides isolated polynucleotides comprising a SNP located within a sequence selected from the group consisting of sequences identified by SEQ. ID. NOS.:1-7 and the complements of sequences identified by SEQ. ID. NOS.:1-7; wherein the presence of a particular allele of a SNP (a particular nucleotide base) is indicative of a propensity to develop Type 2 diabetes or otherwise may be used to identify a Type 2 diabetic. In one embodiment, the polynucleotide is selected from the group consisting of sequences identified by SEQ. ID. NOS.:1-7 and the complements of sequences identified by SEQ. ID. NOS.:1-7. In another embodiment, the polynucleotide comprises at least a portion of a sequence selected from the group consisting of sequences identified by SEQ. ID. NOS.:1-7 and the complements of sequences identified by SEQ. ID. NOS.:1-7.

The present invention also relates to isolated polynucleotides comprising a SNP located within a sequence selected from the group consisting of sequences identified by SEQ. ID. NOS.:1-7 and the complements of sequences identified by SEQ. ID. NOS.:1-7, which hybridize, are complementary, or are partially complementary to a nucleotide sequence present in a test sample. In one embodiment, an isolated polynucleotide is selected from the group consisting of sequences identified by SEQ. ID. NOS.:1-7 and the complements of sequences identified by SEQ. ID. NOS.:1-7, which hybridizes, is complementary, or is partially complementary to a nucleotide sequence present in a test sample. In a further embodiment, an isolated polynucleotide comprises at least a portion of a sequence selected from the group consisting of sequences identified by SEQ. ID. NOS.:1-7 and the complements of sequences identified by SEQ. ID. NOS.:1-7, which hybridizes, is complementary, or is partially complementary to a nucleotide sequence present in a test sample. In certain embodiments, the SNP is located within SEQ ID NO: 1 or the complement of SEQ ID NO: 1. In certain embodiments, the SNP is located within SEQ ID NO: 2 or the complement of SEQ ID NO: 2. In certain embodiments, the SNP is located within SEQ ID NO: 3 or the complement of SEQ ID NO: 3. In certain embodiments, the SNP is located within SEQ ID NO: 4 or the complement of SEQ ID NO: 4. In certain embodiments, the SNP is located within SEQ ID NO: 5 or the complement of SEQ ID NO: 5. In certain embodiments, the SNP is located within SEQ ID NO: 6 or the complement of SEQ ID NO: 6. In certain embodiments, the SNP is located within SEQ ID NO: 7 or the complement of SEQ ID NO: 7.

The present invention also provides isolated polynucleotides comprising one or more haplotypes selected from the group consisting of the haplotypes identified in FIG. 2 which are indicative of a propensity to develop Type 2 diabetes.

In addition, a polynucleotide of the present invention can be isolated using standard molecular biology techniques and the sequence information provided herein. Using all or a portion of a sequence selected from the group consisting of sequences identified by SEQ. ID. NOS.:1-7 and the complements of sequences identified by SEQ. ID. NOS.:1-7, polynucleotides can be isolated using standard hybridization and cloning techniques (e.g., as described in Sambrook et al., eds., Molecular Cloning: A Laboratory Manual, 2^{nd} ed., Cold Spring Harbor Laboratory, Cold Spring Harbor Laboratory Press, Cold Spring Harbor, N.Y, 1989).

A polynucleotide can be amplified using cDNA, mRNA or genomic DNA as a template and appropriate oligonucleotide primers according to standard PCR amplification techniques. The polynucleotide so amplified can be cloned into an appropriate vector and characterized by DNA sequence analysis. Furthermore, oligonucleotides corresponding to all or a portion of a polynucleotide can be prepared by standard synthetic techniques, e.g., using an automated DNA synthesizer.

Probes based on the sequence of a polynucleotide of the invention can be used to detect transcripts or genomic sequences. A probe may comprise a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding a protein in a sample of cells from a subject, e.g., detecting mRNA levels or determining whether a gene encoding a protein has been mutated or deleted.

In certain embodiments, the invention also provides polypeptides encoded by a polynucleotide, wherein the polynucleotide comprises a SNP located within a sequence selected from the group consisting of sequences identified by SEQ. ID. NOS. 1-7 and the complements of sequences identified by SEQ. ID. NOS. 1-7. In one embodiment, a polypeptide is encoded by a polynucleotide, wherein the polynucleotide is selected from the group consisting of sequences identified by SEQ. ID. NOS.:1-7 and the complements of sequences identified by SEQ. ID. NOS.:1-7. In another embodiment, a polypeptide is encoded by a polynucleotide, wherein the polynucleotide comprises at least a portion of the sequence selected from the group consisting of sequences identified by SEQ. ID. NOS.:1-7 and the complements of sequences identified by SEQ. ID. NOS.:1-7. Also contemplated are antibodies that bind such polypeptides.

The present invention also provides polypeptides encoded by a polynucleotide, wherein the polynucleotide comprises a haplotype selected from the group consisting of the haplotypes identified in FIG. 2.

In certain embodiments, the invention also provides a vector comprising a haplotype identified in FIG. 2 or a SNP located within a sequence selected from the group consisting of the sequences identified by SEQ. ID. NOS. 1-7 and the complements of sequences identified by SEQ. ID. NOS. 1-7; operably linked to a regulatory sequence. In one embodiment, a vector comprises a sequence selected from the group consisting of 10 sequences identified by SEQ. ID. NOS.:1-7 and the complements of sequences identified by SEQ. ID. NOS.: 1-7; operably linked to a regulatory sequence. In another embodiment, a vector comprises at least a portion of a sequence selected from the group consisting of sequences identified by SEQ. ID. NOS.:1-7 and the complements of sequences identified by SEQ. ID. NOS.: 1-7; operably linked to a regulatory sequence.

In certain embodiments, the invention also provides recombinant host cells comprising such vectors. In certain embodiments, the invention also provides a method for producing a polypeptide encoded by a polynucleotide, wherein the polynucleotide comprises a haplotype identified in FIG. 2 or a SNP located within a sequence selected from the group consisting of sequences identified by SEQ. ID. NOS. 1-7 and the complements of sequences identified by SEQ. ID. NOS. 1-7, comprising culturing a recombinant host cell containing such a polynucleotide under conditions suitable for expression. In one embodiment, a polypeptide is produced by culturing a recombinant host cell containing a polynucleotide under conditions for expression, wherein the polynucleotide comprises a sequence selected from the group consisting of sequences identified by SEQ. ID. NOS.:1-7 and the complements of sequences identified by SEQ. ID. NOS.:1-7. In another embodiment, a polypeptide is produced by culturing a recombinant host cell containing a polynucleotide under conditions for expression, wherein the polynucleotide comprises a portion of a sequence selected from the group consisting of sequences identified by SEQ. ID. NOS.:1-7 and the complements of sequences identified by SEQ. ID. NOS.:1-7.

Further contemplated by the invention is a transgenic animal containing a polynucleotide comprising a haplotype identified in FIG. 2 or a SNP located within a sequence selected from the group consisting of sequences identified by SEQ. ID. NOS. 1-7 and the complements of sequences identified by SEQ. ID. NOS. 1-7. In one embodiment, a transgenic animal contains a polynucleotide comprising a sequence selected from the group consisting of sequences identified by SEQ. ID. NOS.:1-7 and the complements of sequences identified by SEQ. ID. NOS.:1-7. In another embodiment, a transgenic animal contains a polynucleotide comprising at least a portion of a sequence selected from the group consisting of sequences identified by SEQ. ID. NOS.:1-7 and the complements of sequences identified by SEQ. ID. NOS.:1-7.

In other embodiments, compositions and kits are contemplated which contain the polynucleotides, proteins, antibodies, vectors, and/or host cells of the present invention.

One application of the current invention involves prediction of those at higher risk of developing Type 2 diabetes. Diagnostic tests that define genetic factors contributing to Type 2 diabetes may be used together with, or independent of, the known clinical risk factors to define an individual's risk relative to the general population. Means for identifying those individuals at risk for Type 2 diabetes should lead to better prophylactic and treatment regimens, including more aggressive management of the current clinical risk factors. In certain embodiments, the present invention includes methods of diagnosing a susceptibility to Type 2 diabetes in an individual, comprising detecting polymorphisms in nucleic acids of specific genes or gene segments, wherein the presence of the polymorphism in the nucleic acid is indicative of a susceptibility to Type 2 diabetes.

In certain embodiments, the present invention includes methods of diagnosing Type 2 diabetes or a susceptibility to Type 2 diabetes in an individual, comprising determining the presence or absence of particular alleles of SNPs contained in SEQ. ID. NOS. 1-7 and shown in FIG. 1. In one aspect of the invention, methods comprise screening for one of the at-risk alleles associated with Type 2 diabetes shown in FIG. 1. In certain embodiments, the SNP is located within SEQ ID NO: 1 or the complement of SEQ ID NO: 1. In certain embodiments, the SNP is located within SEQ ID NO: 2 or the complement of SEQ ID NO: 2. In certain embodiments, the SNP is located within SEQ ID NO: 3 or the complement of SEQ ID NO: 3. In certain embodiments, the SNP is located within SEQ ID NO: 4 or the complement of SEQ ID NO: 4. In certain embodiments, the SNP is located within SEQ ID NO: 5 or the complement of SEQ ID NO: 5. In certain embodiments, the SNP is located within SEQ ID NO: 6 or the complement of SEQ ID NO: 6. In certain embodiments, the SNP is located within SEQ ID NO: 7 or the complement of SEQ ID NO: 7.

In one embodiment, the invention provides a method of detecting the presence of a polynucleotide in a sample containing a SNP located within a sequence selected from the group consisting of sequences identified by SEQ. ID. NOS. 1-7 and the complements of sequences identified by SEQ. ID. NOS. 1-7, wherein the method comprises contacting the sample with an isolated polynucleotide comprising a sequence (or a portion of a sequence) selected from the group consisting of sequences identified by SEQ. ID. NOS.:1-7 and the complements of sequences identified by SEQ. ID. NOS.:1-7, under conditions appropriate for hybridization, and assessing whether hybridization has occurred between the polynucleotide in the sample and the isolated polynucleotide; wherein if hybridization has occurred, a certain polynucleotide containing a particular allele of a SNP associated (or not associated) with Type 2 diabetes is present in the sample. In certain embodiments of the above method, the isolated polynucleotide is completely complementary to the polynucleotide present in the sample. In other embodiments of the above method, the isolated polynucleotide is partially complementary to the polynucleotide present in the sample. In other embodiments, the isolated polynucleotide is at least 80% identical to the polynucleotide present in the sample and capable of selectively hybridizing to said polynucleotide. If desired, amplification of the polynucleotide present in the sample can be performed using known methods in the art.

The present invention further provides a method for assaying a sample for the presence of a first polynucleotide which is at least partially complementary to a part of a second polynucleotide wherein the second polynucleotide comprises a sequence selected from the group consisting of sequences identified by SEQ. ID. NOS.:1-7 and the complements of sequences identified by SEQ. ID. NOS.:1-7 comprising: a) contacting said sample with said second polynucleotide under conditions appropriate for hybridization, and b) assessing whether hybridization has occurred between said first and said second polynucleotide, wherein if hybridization has occurred, said first polynucleotide is present in said sample. In one embodiment of the method hereinbefore described, the presence of said first polynucleotide is indicative of Type 2 diabetes or the propensity to develop Type 2 diabetes. In a further embodiment of said method, said second polynucleotide is completely complementary to a part of the sequence of said first polynucleotide. In another embodiment, said method further comprises amplification of at least part of said first polynucleotide. In a further embodiment, said second polynucleotide is 99 or fewer nucleotides in length and is either: (a) at least 80% identical to a contiguous sequence of nucleotides in said first polynucleotide or (b) capable of selectively hybridizing to said first polynucleotide.

Also contemplated by the invention is a method of assaying a sample for the presence of a polypeptide associated with Type 2 diabetes encoded by a polynucleotide, wherein the polynucleotide comprises an allele of a SNP associated with Type 2 diabetes located within a sequence selected from the group consisting of sequences identified by SEQ. ID. NOS. 1-7 and the complements of sequences identified by SEQ. ID. NOS. 1-7, the method comprising contacting the sample with an antibody that specifically binds to said polypeptide. In one embodiment, the presence of a polypeptide associated with Type 2 diabetes in a sample encoded by a polynucleotide (comprising a sequence selected from the group consisting of sequences identified by SEQ. ID. NOS.:1-7 and the complements of sequences identified by SEQ. ID. NOS.:1-7) is assayed by contacting the sample with an antibody that specifically binds to said polypeptide. In another embodiment, the presence of a polypeptide associated with Type 2 diabetes in a sample encoded by a polynucleotide (comprising at least a portion of a sequence selected from the group consisting of sequences identified by SEQ. ID. NOS.:1-7 and the complements of sequences identified by SEQ. ID. NOS.:1-7) is assayed by contacting the sample with an antibody that specifically binds to said polypeptide.

The present invention also includes a reagent for assaying a sample for the presence of a first polynucleotide comprising a SNP located within a sequence selected from the group consisting of sequences identified by SEQ ID. NOS.: 1-7 and the complements of sequences identified by SEQ ID. NOS.: 1-7, said reagent comprising a second polynucleotide comprising a contiguous nucleotide sequence which is at least partially complementary to a part of the first polynucleotide. In one embodiment of said reagent, said second polynucleotide is completely complementary to a part of the first polynucleotide.

The present invention also encompasses a reagent kit for assaying a sample for the presence of a first polynucleotide comprising a SNP located within a sequence selected from the group consisting of sequences identified by SEQ ID. NOS.: 1-7 and the complements of sequences identified by SEQ ID. NOS: 1-7, comprising in separate containers: a) one or more labeled second polynucleotides comprising a sequence selected from the group consisting of the sequences identified by SEQ ID. NOS.: 1-7 and the complements of sequences identified by SEQ ID. NOS.: 1-7; and b) reagents for detection of said label.

In other embodiments, kits are contemplated containing polynucleotides which can be used to assay samples for the presence of polynucleotides containing an allele of a SNP associated (or not associated) with Type 2 diabetes located within a sequence selected from the group consisting of sequences identified by SEQ. ID. NOS. 1-7 and the complements of sequences identified by SEQ. ID. NOS. 1-7. Kits are also contemplated which contain antibodies which can be used to assay samples for the presence of proteins associated (or not associated) with Type 2 diabetes that are encoded by the polynucleotides containing an allele of a SNP associated (or not associated) with Type 2 diabetes.

Other methods of diagnosing a susceptibility to Type 2 diabetes in an individual comprise determining the expression or composition of a polypeptide in a control sample encoded by a polynucleotide containing an allele of a SNP not associated with Type 2 diabetes and comparing it with the expression or composition of a polypeptide in a test sample encoded by the same polynucleotide except containing an allele of a SNP associated with Type 2 diabetes, wherein the presence of an alteration in expression or composition of the polypeptide in the test sample compared to the control sample is indicative of a susceptibility to Type 2 diabetes.

In certain embodiments, the invention also relates to a method of diagnosing Type 2 diabetes or a susceptibility to Type 2 diabetes in an individual, comprising determining the presence or absence in the individual of certain haplotypes. In one aspect of the invention, methods comprise screening for one of the at-risk haplotypes shown in FIG. 2. Thus, the present invention encompasses a method for diagnosing a susceptibility to Type 2 diabetes in an individual, or a method of screening for individuals with a susceptibility to Type 2 diabetes, comprising detecting a haplotype associated with Type 2 diabetes selected from the group consisting of the haplotypes shown in FIG. 2.

The presence or absence of the haplotype may be determined by various methods, including, for example, using enzymatic amplification of nucleic acid from the individual, electrophoretic analysis, restriction fragment length polymorphism analysis and/or sequence analysis.

A method of diagnosing a susceptibility to Type 2 diabetes in an individual, or for screening individuals for a susceptibility to Type 2 diabetes is also included, comprising: a) obtaining a polynucleotide sample from said individual; and b) analyzing the polynucleotide sample for the presence or absence of a haplotype, comprising a haplotype shown in FIG. 2, wherein the presence of the haplotype corresponds to a susceptibility to Type 2 diabetes.

In certain embodiments, a method of determining the susceptibility to Type 2 diabetes in an individual is provided comprising detecting multiple SNPs identified in FIG. 1 or 2. In certain embodiments, the method of determining the susceptibility to Type 2 diabetes in an individual comprises detecting multiple SNPs identified in SEQ. ID. NOS.: 1, 2, 3 and/or 4. In other embodiments, the method of determining the susceptibility to Type 2 diabetes in an individual comprises detecting multiple SNPs identified in SEQ. ID. NOS.: 4, 5, 6, and/or 7. In other embodiments, the method of determining the susceptibility to Type 2 diabetes in an individual comprises detecting multiple SNPs identified in SEQ. ID. NOS.: 1,2, 6, and/or 7. In other embodiments, the method of determining the susceptibility to Type 2 diabetes in an individual comprises detecting multiple SNPs identified in SEQ. ID. NOS.:3, 4, 5, and/or 6. In certain embodiments, the presence of a first polynucleotide in a sample containing one or more at-risk alleles in FIG. 1 is assayed for by contacting the sample with probe polynucleotides that are complementary to said first polynucleotide. In certain embodiments, at least one SNP is located within SEQ ID NO: 1 or the complement of SEQ ID NO: 1. In certain embodiments, at least one SNP is located within SEQ ID NO: 2 or the complement of SEQ ID NO: 2. In certain embodiments, at least one SNP is located within SEQ ID NO: 3 or the complement of SEQ ID NO: 3. In certain embodiments, at least one SNP is located within SEQ ID NO: 4 or the complement of SEQ ID NO: 4. In certain embodiments, at least one SNP is located within SEQ ID NO: 5 or the complement of SEQ ID NO: 5. In certain embodiments, at least one SNP is located within SEQ ID NO: 6 or the complement of SEQ ID NO: 6. In certain embodiments, at least one SNP is located within SEQ ID NO: 7 or the complement of SEQ ID NO: 7.

In certain methods of the invention, a Type 2 diabetes therapeutic agent is contemplated. The Type 2 diabetes therapeutic agent can be an agent that alters (e.g., enhances or inhibits) polypeptide activity and/or expression of a polynucleotide comprising a haplotype identified in FIG. 2 or a SNP located within a sequence selected from the group consisting of sequences identified by SEQ. ID. NOS.: 1-7 and the complements of sequences identified by SEQ. ID. NOS.: 1-7. Such agents include polynucleotides, polypeptides, receptors, binding agents, peptidomimetics, fusion proteins, prodrugs, antibodies, agents that alter polynucleotide expression, agents that alter activity of a polypeptide encoded by a gene or polynucleotide of the invention, agents that alter post-transcriptional processing of a polypeptide encoded by a gene or polynucleotide of the invention, agents that alter interaction of a polypeptide with a binding agent or receptor, agents that alter transcription of splicing variants encoded by a gene or polynucleotide, and ribosomes. In certain embodiments, the invention also relates to pharmaceutical compositions comprising at least one of the Type 2 diabetes therapeutic agents as described herein.

Type 2 diabetes therapeutic agents can alter polypeptide activity or expression of a polynucleotide by a variety of means, such as, for example, by upregulating the transcription or translation of the polynucleotide encoding the polypeptide, by altering posttranslational processing of the polypeptide, by altering transcription of splicing variants, or by interfering with polypeptide activity (e.g., by binding to the polypeptide, or by binding to another polypeptide that interacts with the polypeptide of interest) by downregulating the expression, transcription or translation of a polynucleotide encoding the polypeptide, or by altering interaction among the polypeptide of interest and a polypeptide binding agent.

In certain embodiments, the invention also pertains to a method of treating an individual suffering from Type 2 diabetes by administering a Type 2 diabetes therapeutic agent to the individual in a therapeutically effective amount. In certain embodiments, the Type 2 diabetes therapeutic agent is an agonist and, in other embodiments, the Type 2 diabetes therapeutic agent is an antagonist In certain embodiments, the invention additionally pertains to the use of a Type 2 diabetes therapeutic agent for the manufacture of a medicament for use in the treatment of Type 2 diabetes.

The therapeutic agents as described herein can be delivered in a composition or alone. They can be administered systemically, or can be targeted to a particular tissue. The therapeutic agents can be produced by a variety of means, including chemical synthesis; recombinant production and in vivo production (e.g., a transgenic animal, see U.S. Pat. No. 4,873,316 to Meade et al., incorporated herein by reference in its entirety), and can be isolated using standard methods known in the art. In addition, a combination of any of the above methods of treatment (e.g., administration of a polypeptide in conjunction with antisense therapy targeting mRNA; administration of a first splicing variant in conjunction with antisense therapy targeting a second splicing variant) can also be used.

In certain embodiments, the current invention also encompasses methods of monitoring the effectiveness of therapeutic agents of the invention on the treatment of Type 2 diabetes using methods known in the art. Another application of the current invention is its use to predict an individual's response to a particular therapeutic agent. For example, SNPs or haplotypes may be used as a pharmacogenomic diagnostic to predict drug response and guide the choice of therapeutic agent in a given individual.

In other embodiments, the invention pertains to a method of identifying an agent that alters expression of a polynucleotide containing an allele of a SNP associated with Type 2 diabetes comprising: (a) contacting a polynucleotide with an agent to be tested under conditions for expression, wherein the polynucleotide comprises, (1) an allele of a SNP associated with Type 2 diabetes located within a sequence selected from the group consisting of sequences identified by SEQ. ID. NOS. 1-7 and the complements of sequences identified by SEQ. ID. NOS. 1-7, and (2) a promoter region operably linked to a reporter gene; (b) assessing the level of expression of the reporter gene in the presence of the agent; (c) assessing the level of expression of the reporter gene in the absence of the agent; and (d) comparing the level of expression in step (b) with the level of expression in step (c) for differences indicating that expression was altered by the agent.

In other embodiments, the invention pertains to a method of identifying an agent suitable for treating Type 2 diabetes comprising: (a) contacting a polynucleotide with an agent to be tested, wherein the polynucleotide contains a haplotype identified in FIG. 2 or a SNP located within a sequence selected from the group consisting of sequences identified by SEQ. ID. NOS. 1-7 and the complements of sequences identified by SEQ. ID. NOS. 1-7; and (b) determining whether said agent binds to, alters, or affects the polynucleotide in a manner which would be useful for treating Type 2 diabetes.

In certain embodiments, the expression of the polynucleotide in the presence of the agent comprises expression of one or more splicing variant(s) that differ in kind or in quantity from the expression of one or more splicing variant(s) in the absence of the agent.

In other embodiments, the invention pertains to a method of identifying an agent suitable for treating Type 2 diabetes comprising: (a) contacting a polypeptide with an agent to be tested, wherein the polypeptide is encoded by a polynucleotide containing a haplotype identified in FIG. 2 or a SNP located within a sequence selected from the group consisting of sequences identified by SEQ. ID. NOS. 1-7 and the complements of sequences identified by SEQ. ID. NOS. 1-7; and (b) determining whether said agent binds to, alters, or affects the polypeptide in a manner which would be useful for treating Type 2 diabetes. Agents identified by the above methods are also contemplated as well as pharmaceutical compositions containing such agents.

In one embodiment, a polynucleotide comprising a haplotype identified in FIG. 2 or a SNP located within a sequence selected from the group consisting of sequences identified by SEQ. ID. NOS.: 1-7 and the complements of sequences identified by SEQ. ID. NOS.: 1-7 is used in “antisense” therapy in which the polynucleotide is administered or generated in situ and specifically hybridizes to mRNA and/or genomic DNA. The antisense polynucleotide that specifically hybridizes to the mRNA and/or DNA inhibits expression of the polypeptide encoded by that mRNA and/or DNA, e.g., by inhibiting translation and/or transcription. Binding of the antisense polynucleotide can be by conventional base pair complementarity, or, for example, in the case of binding to DNA duplexes, through specific interaction in the major groove of the double helix.

An antisense construct can be delivered, for example, as an expression plasmid. When the plasmid is transcribed in the cell, it produces RNA that is complementary to a portion of the mRNA and/or DNA that encodes a polypeptide. Alternatively, the antisense construct can be a polynucleotide probe that is generated ex vivo and introduced into cells; it then inhibits expression by hybridizing with the mRNA and/or genomic DNA encoding the polypeptide. In one embodiment, the polynucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996, 5,264,564, and 5,256,775, all of which are incorporated herein by reference in their entirety). Additionally, general approaches to constructing oligomers useful in antisense therapy are also described, for example, by Van der Krol et al. (BioTechniques 6:958-976 (1988)); and Stein et al. (Cancer Res. 48:2659-2668 (1988)), both of which are incorporated herein by reference in their entirety. With respect to antisense DNA, oligodeoxyribonucleotides derived from the translation initiation site may be used.

To perform antisense therapy, oligonucleotides are designed that are complementary to MRNA encoding a polypeptide. The antisense oligonucleotides bind to mRNA transcripts and prevent translation. Absolute complementarity, is not required as along as the oligonucleotides have sufficient complementarity to be able to hybridize with the RNA, forming a stable duplex. The ability to hybridize will depend on both the degree of complementarity and the length of the antisense oligonucleotides. Generally, the longer the hybridizing oligonucleotides, the more base mismatches with RNA they may contain and still form a stable duplex (or triplex, as the case may be). One skilled in the art can ascertain a tolerable degree of mismatch by the use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane (see, e.g., Letsinger et al, Proc. Natl. Acad. Sci. USA 86:6553-6556 (1989); Lemaitre et al., Proc. Natl. Acad. Sci USA 84:648-652 (1987); PCT International Publication NO: WO 88/09810) or the blood-brain barrier (see, e.g., PCT International Publication NO: WO 89/10134), or hybridization-triggered cleavage agents (see, e.g., Krol et al. Bio Techniques 6:958-976 (1988)) or intercalating agents. (See, e.g., Zon, Pharm. Res. 5:539-549 (1988)). To this end, the oligonucleotide may be conjugated to another molecule (e.g., a peptide, hybridization triggered cross-linking agent, transport agent, hybridization-triggered cleavage agent).

In certain embodiments, the antisense molecules are delivered to cells that express polypeptides implicated in Type 2 diabetes in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology and methods standard in the art. For example, a plasmid, cosmid or viral vector can be used to prepare the recombinant DNA construct that can be introduced directly into the tissue site. Alternatively, viral vectors can be used which selectively infect the desired tissue, in which case administration may be accomplished by another route (e.g., systemically). In another embodiment of the invention, small double-stranded interfering RNA (RNA interference (RNAi)) can be used. RNAI is a post-transcription process, in which double-stranded RNA is introduced, and sequence-specific gene silencing results, though catalytic degradation of the targeted MRNA. See, e.g., Elbashir, S. M. et al. Nature 411:494-498 (2001); Lee, N. S., Nature Biotech. 19:500-505 (2002); Lee, S-K. et al., Nature Medicine 8(7):681-686 (2002); the entire teachings of which are incorporated herein by reference in their entirety.

In one embodiment, the invention comprises a short interfering nucleic acid (“siNA”) molecule comprising a double-stranded RNA polynucleotide that down-regulates expression of a polynucleotide containing a haplotype identified in FIG. 2 or a SNP identified in a sequence selected from the group consisting of sequences identified by SEQ. ID. NOS.: 1-7 and the complements of sequences identified by SEQ. ID. NOS.: 1-7. In other embodiments, the invention comprises polynucleotides, compositions, and methods used in RNA interference (as described in U.S. patent Publication NOS: US 2004/0192626 A1, US 2004/0203145 A1, and US 2004/0198682 A1 [all of which are incorporated herein by reference in their entirety]) to alter the expression of genes containing a SNP identified in a sequence selected from the group consisting of sequences identified by SEQ. ID. NOS.: 1-7 and the complements of sequences identified by SEQ. ID. NOS.: 1-7.

Endogenous expression of a gene product can also be reduced by inactivating or “knocking out” the gene or its promoter using targeted homologous recombination. For example, an altered, non-functional gene (or a completely unrelated DNA sequence) flanked by DNA homologous to the endogenous gene (either the coding regions or regulatory regions of the gene) can be used, with or without a selectable marker and/or a negative selectable marker, to transfect cells that express the gene in vivo. Insertion of the DNA construct, via targeted homologous recombination, results in inactivation of the gene. The recombinant DNA constructs can be directly administered or targeted to the required site in vivo using appropriate vectors, as described above. Alternatively, expression of non-altered genes can be increased using a similar method: targeted homologous recombination can be used to insert a DNA construct comprising a non-altered functional gene, or the complement thereof, or a portion thereof, in place of a gene in the cell, as described above. In another embodiment, targeted homologous recombination can be used to insert a DNA construct comprising a polynucleotide that encodes a polypeptide variant that differs from that present in the cell.

Alternatively, endogenous expression of a gene product can be reduced by targeting deoxyribonucleotide sequences complementary to the regulatory region (i.e., the promoter and/or enhancers) to form triple helical structures that prevent transcription of the gene in target cells in the body. (See generally, Helene, C., Anticancer Drug Des., 6(6):569-84 (1991); Helene, C., et al., Ann. N.Y. Acad. Sci. 660:27-36 (1992); and Maher, L. J., Bioassays 14(12):807-15 (1992)); all of which are incorporated herein by reference in their entirety. Likewise, the antisense constructs described herein can be used in the manipulation of tissue by antagonizing the normal biological activity of the gene product, e.g., tissue differentiation, both in vivo and for ex vivo tissue cultures. Furthermore, the anti-sense techniques (e.g., microinjection of antisense molecules, or transfection with plasmids whose transcripts are anti-sense with regard to RNA or nucleic acid sequences) can be used to investigate the role of one or more genes involved in the pathway involved in the development of Type 2 diabetes and related conditions. Such techniques can be utilized in cell culture, but can also be used in the creation of transgenic animals.

The polynucleotides, proteins, and/or therapeutic agents of the invention described herein can be incorporated into pharmaceutical compositions suitable for administration. Such compositions typically comprise polynucleotides, proteins, and/or therapeutic agents and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELTM (BASF; Parsippany, N.J.) or phosphate buffered saline (PBS). In some cases, the composition is sterile and should be fluid to the extent that easy syringability exists. It must be stable under the conditions of manufacture and storage and must be preserved against the contaminating action of microorganisms such as bacteria and fingi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polynucleotide, polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, some methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to the achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl Acad. Sd. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration. Kits are contemplated which contain the therapeutic agents of the invention.

Another embodiment of the invention is its use to predict an individual's response to a particular drug to treat Type II diabetes. It is a well-known phenomenon that in general, patients do not respond equally to the same drug. Much of the differences in drug response to a given drug is thought to be based on genetic and protein differences among individuals in certain genes and their corresponding pathways. The present invention defines particular SNPs, haplotypes, and genes that are associated with Type 2 diabetes. Some current or future therapeutic agents may be able to affect pathways that are related to such SNPs, haplotypes, and/or genes directly or indirectly and therefore, be effective in those patients whose Type II diabetes risk is in part determined by such SNPs, haplotypes, and/or genes. On the other hand, those same drugs may be less effective or ineffective in those patients who do not have particular alleles of said SNPs and/or haplotypes. Therefore, the SNPs and/or haplotypes of the present invention may be used as a pharmacogenomic diagnostic to predict drug response and guide choice of therapeutic agent in a given individual.

In one embodiment, a method for monitoring the effectiveness of a drug on the treatment of Type 2 diabetes comprises, monitoring the level of expression of a gene associated with Type 2 diabetes containing one or more SNPs selected from the group of SNPs consisting of the SNPs identified in FIG. 1 before treatment with a drug, monitoring the expression of said gene after treatment with said drug, and comparing the level of expression of said gene before said treatment and after said treatment.

In another embodiment, a method for predicting the effectiveness of a given therapeutic agent in the treatment of Type 2 diabetes comprises screening for the presence or absence of one or more SNPs located within a sequence selected from the group consisting of sequences identified by SEQ. ID. NOS. 1-7 and the complements of sequences identified by SEQ. ID. NOS. 1-7.

In another embodiment, a method for predicting the effectiveness of a given therapeutic agent in the treatment of Type 2 diabetes comprises screening for the presence or absence of one or more haplotypes identified in FIG. 2.

Another application of the current invention is the specific identification of a rate-limiting pathway involved in Type 2 diabetes. A disease gene with genetic variation that is significantly more common in diabetic patients as compared to controls represents a specifically validated causative step in the pathogenesis of Type 2 diabetes. That is, the uncertainty about whether a gene is causative or simply reactive to the disease process is eliminated. The protein encoded by the disease gene defines a rate-limiting molecular pathway involved in the biological process of Type 2 diabetes predisposition. The proteins encoded by such Type 2 genes or its interacting proteins in its molecular pathway may represent drug targets that may be selectively modulated by small molecule, protein, antibody, or nucleic acid therapies. Such specific information is greatly needed since the population affected with Type 2 diabetes is growing.

Genes not known to be previously implicated with Type 2 diabetes by SNP based association but which were discovered to be implicated with Type 2 diabetes by SNP based association in the present invention include the following:

TABLE 1
Genes Discovered To Be Implicated In Type
2 Diabetes By SNP Based Association

| Gene | Description | Chr | LocusLink |
|---|---|---|---|
| GPC1 | glypican 1 | 2 | 2817 |
| ROBO1 | roundabout, axon guidance receptor, | 3 | 6091 |
| | homolog 1 (Drosophila) | | |
| ROBO2 | roundabout, axon guidance receptor, | 3 | 6092 |
| | homolog 2 (Drosophila) | | |
| KCNIP2 | Kv channel interacting protein 2 | 10 | 30819 |
| ROBO4 | roundabout homolog 4, magic | 11 | 54538 |
| | roundabout (Drosophila) | | |

In one embodiment, the invention pertains to a method of identifying a gene associated with Type 2 diabetes comprising: (a) identifying a gene containing a SNP that is located within a sequence selected from the group consisting of sequences identified by SEQ. ID. NOS. 1-7 and the complements of sequences identified by SEQ. ID. NOS. 1-7; and (b) comparing the expression of said gene in an individual having the at-risk allele with the expression of said gene in an individual having the non-risk allele for differences indicating that the gene is associated with Type 2 diabetes.

In another embodiment, the invention pertains to a method of identifying a gene associated with Type 2 diabetes comprising: (a) identifying a gene containing an at-risk haplotype identified in FIG. 2; and (b) comparing the expression of said gene in an individual having the at-risk haplotype with the expression of said gene in an individual not having the at-risk haplotype for differences indicating that the gene is associated with Type 2 diabetes.

### EXAMPLE 1

### Study Populations

A total of 600 patients were included in the study to investigate the variation in the presence of SNPs and SNP haplotypes as between control and Type 2 diabetes populations. One cohort of Swedish diabetics was used for SNP discovery and a separate cohort of Polish diabetics was used in the association study (See Table 2). Samples from an additional three hundred matched controls were analyzed for use in the association study.

TABLE 2
Summary of case and control samples used in this study.

| | Ethnicity (Country of | | |
|---|---|---|---|
| Disease Status | Origin) | Number | Usage |
| Diabetic Cases | Caucasian (Sweden) | 300 | SNP Discovery |
| Diabetic Cases | Caucasian (Poland) | 300 | SNP Genotyping |
| Unaffected Controls | Caucasian (Poland) | 300 | SNP Genotyping |

From the case-control study, the phenotype was simply “diabetes”. Other sub-phenotypes could be included in the analysis including BMI, haemoglobin AIC, heart disease (MI etc), nephropathy etc. The samples were collected by Genomics Collaborative Inc. (CGI) according to protocols detailed in Ardlie et al., Testing for population subdivision and association in four case-control populations, Am J Hum Genet. 71:304-311, 2002, incorporated herein by reference in its entirety.

Finally, the population contained roughly equal numbers of males and females (274 males and 326 females). Samples were also well matched with identical numbers of males (163 cases and 163 controls) and females (137 cases and 137 controls) in the diabetic and unaffected groups.

In any population based study, it is important to match the cases and controls in order to avoid spurious results based on unknown, confounding factors. In the context of genetics studies, this means that case and control populations should be genetically identical across the genome, with the exception of regions containing genes that predispose to the phenotype being studied. That is, a random set of markers should show broadly similar allele frequencies in the case and control populations. Population stratification was unlikely to be present in this study as the patients and controls were not only matched for sex and ethnicity, but were selected from the same country (Poland). However, to test for population stratification, the data was analyzed using the software program STRUCTURE 2.0 by Falush et al. (March 2002); see also Pritchard et al, Inference of population structure: Extensions to linked loci and correlated allele frequencies; GENETICS In Press; (2003); Pritchard et al, Inference of population Structure Using Multilocus Genotype Data; GENETICS 155: 945-959 (2000a); all of which are incorporated herein by reference in their entirety. STRUCTURE implements a model-based clustering method as described in Pritchard et al, Association mapping in structured population,. AM. J. HUM. GENET. 671:170-81 (2000); incorporated herein by reference in its entirety. The program was allowed to sort the data into pre-specified numbers of clusters without any intervention. The data sets consisted of 150 markers which were chosen based on three criteria. First, the minor allele had to have frequency>5% in the total population. Second, at least 80% of the individuals were required to have genotypes and third, the markers could not be closer than 100 kb to any other marker in the set.

Stratification analysis of the data showed no clear clustering. A variety of factors indicated a lack of structure including the following:

the proportion of an individual's genome from each of the clusters was the same for cases and controls, all individuals were admixed. That is they were deemed to have genes from all clusters, and the likelihood was not improved by adding more parameters (i.e. fitting more clusters).

In summary, there is not consistent genetic bias between the cases and controls. The best-fit dusters generated by STRUCTURE appear to be unrelated to the phenotype of the individual samples.

### EXAMPLE 2

### Sample Collection and SNP Discovery in Diabetic Population Candidate Genes

Three hundred (300) samples from diabetic patients were analyzed for the SNP discovery process. A total of 186 genes (identified as being implicated in diabetes genes) were utilized for SNP discovery. Of these genes, 62 were analyzed to detect 341 SNPs utilizing ParAllele's Mismatch Repair Detection System (MRD). The other 1659 SNPs were identified by de novo sequencing and identified from public databases (National Center for Biotechnology Information).

Of the 186 genes which were analyzed for SNP discovery, 62 genes were analyzed to detect 341 SNPs via the Mismatch Repair Detection platform (MRD). The aim of the analysis was to discover SNPs in these targets that are at 2% frequency or higher in the study population including diabetic and control populations. Information on the exons of all the genes was taken from Ensembl (The Sanger Centre, Cambridge, UK) database build 33. As the sequences immediately upstream of the transcript are enriched for regulatory sequences, the first 350bp upstream were coded as exon 0. A total of 990 regions were identified where each of the exons, human mouse homologies as well as exon 0 are regions. One hundred and seventy five of these regions were human mouse homologies and 815 were exons (including exon 0). The number of exons per gene ranged from 2 (including exon 0) as in the case for PPPIR3C to 58 (NOS2A). Some of the large exons were divided into two or more targets and some pairs of small closely spaced exons were merged to form one target. In total, 1011 targets were generated, and primers designed to amplify 999 amplicons.

### EXAMPLE 3

### Mismatch Repair Detection (MRD)

MRD detects variants or SNPs utilizing the mismatch repair system of Escherichia coli Modrich, P., Mechanisms and biological effects of mismatch repair, ANN REV. GENET, 25: 2259-53 (1991), incorporated herein by reference in its entirety. A specific strain is engineered to sort a pool of transformed fragments into two pools: those carrying a variation and those that do not. MRD has been described before as a method for multiplex variation scanning Faham. M., et al., Mismatch repair detection (MDRD): high-throughput scanning for DNA variations, HuM MOL. GENET, 10(16);p 1657-64 (2001), incorporated herein by reference in its entirety. MRD is used in combination with standard dideoxy terminator sequencing to discover common variant alleles in two different populations. Individual PCR reactions using pooled genomic DNA from a population as a template are mixed with PCR fragments from a single haploid individual. Sanger sequencing does not have sufficient sensitivity to detect rare alleles from genomic pools in which the pooled population is sequenced directly. Instead, many PCR reactions are pooled and one MRD reaction is done to produce a pool of colonies enriched for variant alleles compared to the haploid standard. One amplication reaction from the variant-enriched pool is done for each amplicon followed by a sequencing reaction to identify common and rare variations in the population examined. See Fakhrai-Rad et al., SNP Discovery in Pooled Samples With Mismatch Repair Detection, COLD SPRING HARBOR LABORATORY PRESS, 14: 1404-1412 (2004), incorporated herein by reference in its entirety.

The end result of this process is that the necessity of ampliing and sequencing many individuals is replaced with a pooled enrichment process that is carried out for thousands of amplicons in a multiplexed fashion. The sequencing process is thus reduced to the task of sequencing a haploid standard and the result of an MRD enriched pool. Amplicons are typically sequenced in both forward and reverse directions to reduce the false positive SNP discovery rate.

The sensitivity of MRD based SNP discovery is limited by backgrounds caused by MRD enrichment of non-genomic DNA mismatches. These can occur in two ways: oligonucleotide mutations and PCR error. Both oligo error in the PCR primers and PCR errors introduce a set of fragments which contain mutations in the absence of any actual DNA variation. These fragments will be enriched along with the actual variations meaning that it is impossible to enrich a mutation that occurs at a frequency lower than the background level. Oligos having low rates of mutation and PCR using high fidelity polymerases are used in order to minimize these problems. Control experiments were performed using patients with variation in the BRCA1 gene. These patients were sequenced to identify mutations in the BRCA1 exons. These DNAs were then pooled in such a way as to create samples in which individual SNPs were found at a range of frequencies. These pools were then enriched and sequenced. MRD displays a very high sensitivity to variations as low as 1% frequency with complete rediscovery in cases in which an amplicon exhibits a SNP at >10% frequency.

In human populations, there are other practical issues which impose other limitations on the sensitivity of MRD-based SNP discovery. The first of these is that multiple SNPs can occur on a particular sequencing fragment. If this occurs with the two SNPs having very different frequencies, the SNP with the higher frequency will tend to dominate the enriched pool, suppressing the signal of the rarer SNP. This effect can be mitigated in several ways. The first is to use fairly small PCR fragments to minimize the chances of multiple SNPs occurring within a single fragment (typically fragments of ˜300 bp are used). Secondly, in cases when common SNPs are known to occur, PCR primers can be designed to exclude these SNPs. These limitations are to be weighed against the prohibitive costs of sequencing and analysis of many individuals in the typical manner. Reducing the number of individuals sequenced in the classical manner reduces coverage by introducing Poisson noise in the choice of a small population.

### EXAMPLE 4

### SNP Genotyping Using Molecular Inversion Probes

Following the completion of the SNP discovery phase utilizing samples from diabetic patients another set of samples including a set of 300 samples from a second cohort of diabetics and a set of 300 samples from non-diabetic controls were utilized for the genotyping phase of the project.

Molecular Inversion Probes (MIP) were utilized for SNP genotyping. Sequences for 1739 SNPs were analyzed. A total of 1591 of these SNPs were unique in the NCB1 database (build 33). The 40 bp sequence flanking 327 of the SNPs were unique. 82/102 validated SNPs from the public databases that were not detected in the SNP discovery were unique in the genome. This gave exactly 2,000 SNPs for which MIP probes were designed. Out of these 2,000 probes, 1,769 (88.4%) yielded validated assays. These were then genotyped in 300 diabetic cases and 300 ethnically and sex matched controls.

These SNPs were chosen to provide information on 186 genes which may play a role in susceptibility to diabetes. These genes are located across the genome with at least one gene from every chromosome with the exception of 21 and the Y chromosome. The genes varied in size from 0 to 992 kb. Note that the length of the gene was measured by size of the region between the most widely spaced SNPs in each gene, hence genes with only one SNP were recorded as having size 0 kb.

The oligonucleotide probes in this process undergo a unimolecular rearrangement from a molecule that cannot be amplified, into a molecule that can be amplified. This rearrangement is mediated by genomic DNA and an enzymatic “gap fill” process that occurs in an allele-specific manner. The gap-fill process results in an important intermediate state in which the probes are circularized. This state allows a selection for the unimolecular interactions through exonuclease treatment that will degrade all cross-reacted and un-reacted probes. After inversion, the probes are amplified using generic PCR primers that are fluorescently labeled. See Hardenbol et al., Multiplexed genotyping with sequence tagged molecular inversion probes, 21 NAT. BIOTECHNOL. (6):673-78 (June, 2003), incorporated herein by reference in its entirety.

In order to identify the allele, four identical reactions are used for the SNPs. Each of four multiplexed reactions scores a different SNP allele by using a single nucleotide species (A, C, G or T). After inversion, PCR is carried out with a common primer pair such that all probes that have undergone inversion will be amplified in each reaction. By using a different fluorescent label in each of these four reactions, the SNP allele can be inferred by identifying which labels are present on the MIP probe amplicon that results from the four separate reactions.

After amplification, the four reactions are hybridized to universal oligonucleotide arrays. The relative base incorporation is measured by the fluorescent signal at the corresponding complementary tag site on the DNA array. Four intensity values for each probe are generated. The two values for the expected allele bases are compared to determine whether the SNP is homozygous or heterozygous for the given individual, and the two non-allele bases are compared to the allele bases to measure the signal to noise for the probe as a quality control check.

### EXAMPLE 5

### Summary of Allelic Association Results

Marker-trait association was examined using contingency table analyses and Fisher's Exact test for empirical p-values. A summary of the results from the allelic chi-square association test (2×2, 1 d.f.) of one particular study are shown in FIG. 3 where a number of SNPs were found to be significant at p≦0.05.

### EXAMPLE 6

### Summary of the Genotypic Association Results

A summary of the results from the genotypic chi-square association test (2×3, 2 d.f.) of one particular study are shown in FIG. 4 where a number of SNPs were found to be significant at p≦0.05.

### EXAMPLE 7

### Chi-Square Tests For Recessive Effects

While both the allele and genotype tests are most appropriate when the underlying genetic liability to disease conforms to an additive genetic model, the genotype test also includes a test for a dominance. However, both the genotype and allele tests do not address recessive allelic effects and if present, they would be missed. To address this problem another series of chi-square tests were run where the minor allele of each SNP was modeled as a recessive effect (2×2, 1 d.f.). Several SNPs were significant by the recessive test (see FIG. 5), some of which were already implicated by the allele test. FIG. 6 provides a summary of the SNPs found to be associated with Type 2 diabetes using allelic association, genotypic association and the chi-square test for recessive effects.

### EXAMPLE 8

### Assessment For At-Risk Haplotypes

The haplotypes described herein are found more frequently in individuals with Type 2 diabetes than in individuals without Type 2 diabetes. Accordingly, these haplotypes have predictive value for detecting Type 2 diabetes or a susceptibility to Type 2 diabetes in an individual. In certain methods described herein, an individual who is at risk for Type 2 diabetes is an individual in whom an at-risk haplotype is identified.

In one embodiment, the at-risk haplotype is one that confers a significant risk of Type 2 diabetes. In one embodiment, significance associated with a haplotype is measured by an odds ratio. In a further embodiment, the significance is measured by a percentage. In one embodiment, a significant risk is measured as an odds ratio of at least about 1.2, including but not limited to: 1.2, 1.3, 1.4, 1.5, 1.6, 1.7, 1.8 and 1.9. In a further embodiment, an odds ratio of at least 1.2 is significant. In a further embodiment, an odds ratio of at least 1.5 is significant. In a further embodiment, a significant increase in risk of at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood, however, that identifying whether a risk is medically significant may also depend on a variety of factors, including the specific disease, the haplotype, and often, environmental factors.

Standard techniques for genotyping for the presence of SNPs can be used, such as fluorescent-based techniques (Chen, et al., Genome Res. 9, 492 (1999)), PCR, LCR, Nested PCR and other techniques for nucleic acid amplification. In one embodiment, the method comprises assessing in an individual the presence or frequency of SNPs, wherein an excess or higher frequency of the SNPs compared to a healthy control individual is indicative that the individual has Type 2 diabetes, or is susceptible to Type 2 diabetes. The presence of two or more SNPs may indicate the presence of an at-risk haplotype that can be used to screen individuals. For example, an at-risk haplotype can include the haplotypes identified in FIG. 2, a combination of SNPs identified in FIG. 1, or a combination of the SNPs identified in FIG. 1 or 2. The presence of an at-risk haplotype is indicative of a susceptibility to Type 2 diabetes, and therefore is indicative of an individual who falls within a target population for the treatment methods described herein.

1. A method of determining a susceptibility to Type 2 diabetes in an individual, comprising detecting an at-risk allele of a SNP associated with Type 2 diabetes, wherein the SNP is located within a sequence selected from the group consisting of sequences identified by SEQ. ID. NOS.: 1-7 and the complements of sequences identified by SEQ. ID. NOS.: 1-7.

2-8. (canceled)

9. An isolated polynucleotide comprising a SNP located within a sequence selected from the group consisting of sequences identified by SEQ. ID. NOS.: 1-7 and the complements of sequences identified by SEQ. ID. NOS.: 1-7.

10-31. (canceled)

32. A method of diagnosing a susceptibility to Type 2 diabetes in an individual, comprising detecting a haplotype associated with Type 2 diabetes selected from the group consisting of the haplotypes shown in FIG. 2.

33-44. (canceled)


SEQUENCE LISTING (7 sequences)

SEQ ID NO: 1 - DNA, 121 bp, Homo sapiens
ccggagtgctcgagagctgtcatgaagctggtctactgtgctcactgcctgggagtccccrgcgccaggccctgccctgactattgccgaaatgtgctcaagggctgccttgccaaccagg

SEQ ID NO: 2 - DNA, 61 bp, Homo sapiens
cccaggctctccatggataccgaggagggtktggaaaatttctggggcatttctaaggaga

SEQ ID NO: 3 - DNA, 61 bp, Homo sapiens
aggtgtatgaatatgttatctgcatttatcygcttatctgtgtatgtgtaaacatctttta

SEQ ID NO: 4 - DNA, 61 bp, Homo sapiens
tgttccacacacacattgaggatcataggcrgatgggttagcctctttatcatggggcttc

SEQ ID NO: 5 - DNA, 61 bp, Homo sapiens
attcttagtgtgtaatcgtctttgatgtcayaactatcaaaggcataattagcactttgac

SEQ ID NO: 6 - DNA, 111 bp, Homo sapiens
tgattgtagcccacccctgcccttaccctggatggaaacccgggctgcgcggctctccctrtgtcctgcgctgttggtggccacacacatgtaggtcccttcgtcactctt

SEQ ID NO: 7 - DNA, 111 bp, Homo sapiens
ggagggaagcggctttgaggaaaccgatgcttcctgcctggccctcagtgccttggagaaytgggaggagaccagaggcgagaaactctctgacctcgaccatttagctat